(19) United States
(12) Patent Application Publication
(10) Pub. No.: US 2003/0050638 A1
(43) Pub. Date: Mar. 13, 2003

(54) Surgical instrument and method of using the same

(76) Inventors: Daniel Yachia, Herzlia-on-Sea (IL); Baruch Levy, Ramat-gan (IL); Zohar Tyroler, Holon (IL); Avishai Chalfon, Shilo (IL); Marvin Slepian, Tuscon, AZ (US)

Correspondence Address: KUDIRKA & JOBSE, LLP, ONE STATE STREET, SUITE 1510, BOSTON, MA, 02109, US

(21) Appl. No.: 09/950,984

(22) Filed: Sep. 12, 2001

(51) Int. Cl. A61B 18/18

(52) U.S. Cl. 606/45

(57) ABSTRACT

There is disclosed to a surgical instrument and methods for its use, for cutting tissue that has a positioning tube having a longitudinal axis, a cutting blade, that is at least substantially continuous, typically a ring, and a shaft. The shaft includes a distal end, that typically extends into the cutting blade, and engages the cutting blade in a rotational engagement, so as to confine its position while allowing it to rotate, upon the shaft rotating. At least a portion of the shaft is housed within the positioning tube. A frame is configured for receiving the proximal end of the shaft such that the shaft can rotate while being held in the frame. This reception and retention of the shaft by the frame is such that movement of the frame along the longitudinal axis moves the shaft and the cutting blade along the longitudinal axis. The cutting blade typically includes a sharpened edge, typically at its proximal end. This instrument is such that the cutting blade can also be used for cauterization of tissue, eliminating any need for separate cutting and cauterizing instruments, thus minimizing the invasiveness of the procedure.

### TECHNICAL FIELD

This invention relates to medical instruments, and more specifically to such instruments used in surgery and minimally invasive therapeutics.

### BACKGROUND

As men age, their prostates (prostate glands) often enlarge due to growth of intraprostatic periurethral gland tissue (prostate adenoma). This condition, known as benign prostatic hypertrophy (BPH), leads to obstruction of urine flow in the urethra resulting in complete, or partial, inability to urinate. The incidence of BPH for men in their fifties is approximately 50%, rising to 90% by age 85. About 25% of men in the United States are treated for BPH by the age of 80.

Various surgical interventions for the treatment of BPH are known in which prostate tissue is excised. These include Transurethral Resection of the Prostate (TURP), Transurethral Incision of the Prostate (TUIP) and Suprapubic or Retropubic (Open) Prostatectomy (SPP/RPP). Of these, the most effective therapy is endoscopic resection of the prostate from within or Transurethral Resection of the Prostate (TURP).

TURP provides the best means of reducing urinary obstruction, though it carries the burden of predominantly being an inpatient procedure. Further, TURP often has post-procedure pain and bleeding and requires the use of post-procedure drainage catheters for an extended period of time. Other limitations include retrograde ejaculation and impotence, and other aspects of sexual dysfunction. While highly effective in reducing an obstruction, the dominant mechanism behind TURP is progressive coring-out of the prostate, beginning at the level of the urethra and progressing radially to the prostatic capsule.

In an attempt to limit hospital stay and patient discomfort, several alternative “less invasive” means of reducing prostatic obstruction have emerged. Many of these methods utilize alternative energy means for removing or destroying prostatic tissue. These include Transurethral Vaporization of the Prostate (TURVP), Visual and Contact Laser Ablation of the Prostate (V-LAP and C-LAP) and TransUrethral Needle Ablation (TUNA). In TUNA, for example, radio-frequency (RF) energy is used to thermally denature or cauterize prostate tissue. In this procedure, one or two RF electrodes are transurethrally inserted into prostatic tissue. Heat generated by the electrodes cauterizes the adjacent prostatic tissues. Despite the advances in urology provided by these new methods, none is as effective as TURP.

Various surgical devices have been used to remove tissue and can be used in the above procedures. Surgical devices known in the art having blades that alternate between a non-cutting position and a cutting position are disclosed in U.S. Pat. Nos. 5,030,201 (Palestrant); U.S. Pat. No. 5,556,408 (Farhat); U.S. Pat. No. 5,154,724 (Andrews); U.S. Pat. No. 5,158,564 (Schnepp-Pesch, et al.); U.S. Pat. No. 5,318,576 (Plassche, Jr., et al.) and U.S. Pat. No. 5,395,311 (Andrews).

Some other devices that perform TURP with RF now detailed. For example, U.S. Pat. No. 5,192,280 (Parins), discloses an RF instrument that contains a pair of bipolar RF electrodes formed in a ceramic head at the end of the instrument. The electrodes lie in the axis of the instrument when being inserted through the urethra into the prostate. The ceramic head is then pivoted to bring the electrodes perpendicular to the axis. Radial incisions are made by applying RF energy across the electrodes from an external power source and drawing the electrodes across prostate gland tissue to cauterize the tissue.

U.S. Pat. No. 5,415,656 (Tihon, et al.) discloses an RF cutter in which the cutter is an electrically conducting loop positioned in a tube. During insertion, the loop is in a non-cutting position within the tube, and is brought into a cutting position by being pushed out of the tube. A disadvantage of this cutter is that a loop-shaped cutter is not ideal for making an incision.

### SUMMARY

The present invention improves on the contemporary art by providing an instrument that allows for the selective removal of tissue, typically from the prostate (prostate gland). The instrument “cold cuts” tissue, as it operates at a surgical site at normal body temperature. The resultant cutting action generates minimal, if any, heat, as it cuts absent RF energy or other heat generated cutting mechanisms, and thus, minimizes the risk of impotence from heat damage to erectile tissue and nerves adjacent to the prostate. The cutting is fine and precise, resulting in sharply cut pieces of tissue, that are suitable for histological examination. As cutting is fine and precise, it occurs without ripping and tearing tissues, that increases unwanted bleeding. Moreover, precision cutting avoids ripped and torn tissue that can wrap around the device, limiting its effectiveness. Additionally, this precision cutting does not cause turbulence at the surgical site, providing the surgeon with a clear view of the surgical site.

The instrument of the invention is such that the cutting blade can also be used for cauterization of tissue, eliminating any need for separate cutting and cauterizing instruments, thus, minimizing the invasiveness of the procedure. The cutting unit of the instrument includes a clear (see-through or transparent) portion, allowing the surgeon a clear view of the surgical site. Additionally, cutting occurs as the cutting blade moves toward the instrument, while irrigating fluid, used to flush the cut tissue is emitted from the instrument so as to flow in a direction away from the instrument. As a result, tissue and other fluids and particulates, resulting from the cutting, are flushed away from the instrument, allowing the physician a continuously clear view of the surgical site.

An embodiment of the invention is directed to a surgical instrument for cutting tissue that has a positioning tube having a longitudinal axis, a cutting blade that is at least substantially continuous, typically a ring, and a shaft, The shaft includes a distal end, that typically extends into the cutting blade, and engages the cutting blade in a rotational engagement, so as to confine its position while allowing it to rotate, upon the shaft rotating. This engagement is typically via a gear with teeth on the shaft, that temporarily engages correspondingly configured portions on the cutting blade. At least a portion of the shaft is housed within the positioning tube. A frame is configured for receiving the proximal end of the shaft such that the shaft can rotate while being held in the frame. This reception and retention of the shaft by the frame is such that movement of the frame along the longitudinal axis moves the shaft and the cutting blade along the longitudinal axis. The cutting blade typically includes a sharpened edge, typically at its proximal end.

Another embodiment of the invention is directed to a method for cutting tissue. This method includes providing a surgical instrument having a tubular sheath having a longitudinal axis, and a rotatable ring defining a cutting blade. The ring is mounted with respect to the tubular sheath so as to be movable in directions along the longitudinal axis between positions inside of the tubular sheath and outside of the tubular sheath. A surgical site is then accessed, and the cutting blade is moved out of the tubular sheath. The cutting blade is then rotated, and while rotating, the cutting blade is moved toward the tubular sheath, resulting in precisely cut tissue pieces.

### BRIEF DESCRIPTION OF THE DRAWINGS

The above and further advantages of the invention may be better understood by referring to the following description in conjunction with the accompanying drawings in which like reference numerals and characters indicate corresponding or like components.

In the drawings:

FIG. 1 is a perspective view of the instrument in accordance with an embodiment of the invention;

FIG. 2 is a cross-sectional view of the instrument of FIG. 1;

FIG. 3 is a cross sectional view of the instrument of FIG. 1 taken along line 3-3;

FIG. 4 is a perspective view of a first embodiment of the cutting unit and its position with respect to the internal tubes of the instrument of FIG. 1, with the tubular sheath removed;

FIG. 5 is a cross sectional view of the instrument of FIG. 1 taken along line 5-5;

FIG. 6 is an enlarged cross-sectional view of FIG. 2;

FIG. 7 is a perspective view of a second embodiment of the cutting unit in accordance with the invention;

FIG. 8 is a perspective view of a third embodiment of the cutting unit in accordance with the invention;

FIG. 9 is a perspective view of an alternate embodiment of the invention with a steerable positioning tube;

FIG. 10 is a perspective view of another alternate embodiment of the invention; and

FIG. 11 is a cross-sectional view of the embodiment of FIG. 10 along line 11-11.

### DETAILED DESCRIPTION OF THE DRAWINGS

The present invention relates to a surgical instrument for cutting tissue. Tissue is typically cut into pieces or the like. While the instrument will be described primarily with reference to prostate (prostate gland) surgery, it should be understood that the instrument might also be used in other forms of minimally invasive surgery or percutaneous therapeutic surgery.

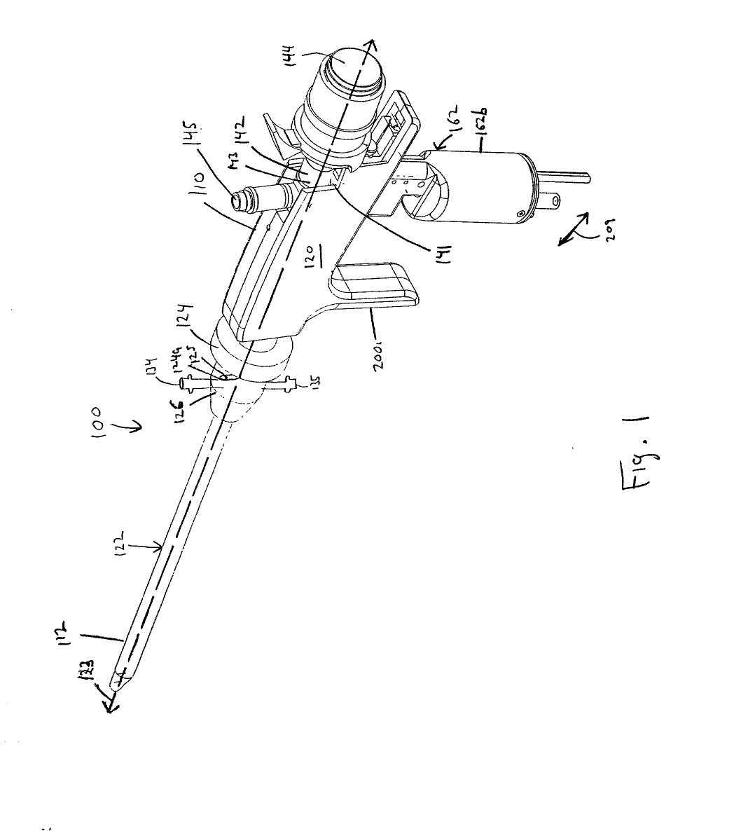

FIGS. 1 and 2 show a surgical instrument 100 in accordance with the invention. This instrument 100 includes a proximal end 110, held by the surgeon, and a distal end 112. The instrument is formed of a body 120, with a tubular sheath 122 extending distally therefrom. The body 120 and tubular sheath 122 define a longitudinal axis 123.

The sheath 122 typically includes an integral shield 124 and is attached to the body 120 by a coupling 126 or the like, that allows the sheath 122 to be removed for sterilization purposes. Specifically, the shield 124 includes slits 124a that engage pins 125 on a nose 126 of the body 120. The slits 124a are configured to securely engage the pins 125 upon the shield 124 being slid onto and rotated (twisted) on the nose 126.

A viewing tube 128, and an instrument tube 130, are within this sheath 122. The space between these tubes 128, 130, defines a lumen 132. Both the viewing tube 128 and instrument tube 130 extend into the body 120, with a fluid-tight seal 133, surrounding the tubes 128, 130, typically in the nose 126, to prevent fluids, e.g., liquids and gasses, and particulates, such as tissue fragments, from entering the body 120 through the lumen 132.

Turning also to FIG. 3, the lumen 132 is formed in the areas of the sheath 122 intermediate the sheath 122 and the viewing 128 and instrument tubes 130. The lumen 132 allows for the transport of fluids or tissue fragments to and from the surgical site (in the case of tissue fragments, from the surgical site). The lumen 132 terminates in the shield 124 at the proximal end 112 of the instrument 100. The shield 124 includes ports 134, 135, in communication with the lumen 132, for receiving fluid supply tubes, suction tubes, outlet tubes, etc. The lumen 132 and ports 134, 135 are also dimensioned to allow for the placement of supply tubes, suction tubes, etc., therethrough, to points distal to the distal end of the sheath 122 and cutting blade unit 160.

The viewing tube 128 is dimensioned to support optics such as viewing devices, including viewing cameras and the like, as detailed in commonly owned U.S. patent application Ser. No. 09/593,988, the disclosure of which is incorporated by reference herein. This viewing tube 128 terminates in a cavity 140 in the body 120. The body 120 includes a receiving area 141, for receiving and supporting a viewing apparatus 142, that is slidably received in the cavity 140 and the viewing tube 128. The viewing apparatus 142 may include a distally extending tube portion 143, a proximal eyepiece 144 and a connection 145. With the requisite optics placed therein, there is formed an endoscopic viewing system, as detailed in U.S. patent application Ser. No. 09/593,988. Additionally, an optical fiber may be provided to the connection 145, to conduct light to the viewing system for illuminating the surgical site, as detailed in U.S. patent application Ser. No. 09/593,988. While these components form an optical system for viewing the surgical site, the aforementioned structures can easily be adapted for other viewing systems including micro-chip cameras, ultrasonic, or the like.

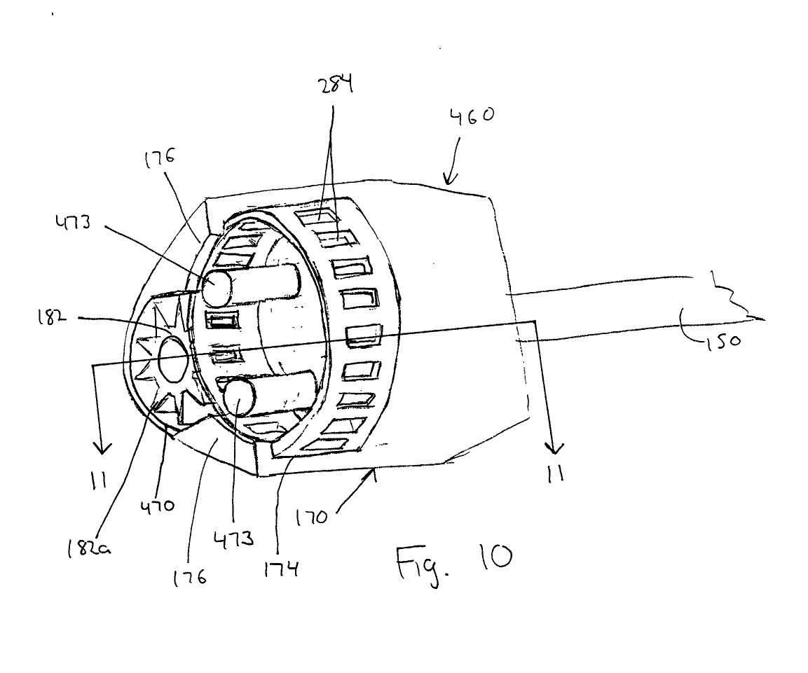

The instrument tube 130 is attached to the body 120 and extends distally therefrom. It houses a portion of a positioning tube 150, that extends through it. This positioning tube 150 is affixed to a cutting unit 160 at its distal end, and at its proximal end, is attached to the frame 162. The positioning tube 150 also houses a shaft 164, that is typically flexible, that extends therethrough. While typically rigid, the positioning tube 150 can also be flexible, and also steerable (FIG. 9, detailed below).

The shaft 164 is affixed to a rotary mechanism within the frame 162 (detailed in FIG. 6 and below) at its proximal end, and it is incorporated into the cutting unit 160 at its distal end. The positioning tube 150 and cutting unit 160 typically extend a slight distance distally from the distal most point of the instrument tube 130, but remain within the sheath 122, when the instrument is in an inactive, or non-cutting position.

Turning also to FIG. 4, there is detailed the cutting unit 160. The cutting unit 160 shown is an example of a cutting unit that can be employed with the present invention. The cutting unit 160 includes a platform 170, whose outer surface 171 is rounded to a curvature sufficient to fit within the curvature of the tubular sheath 122. The platform 170 is typically made of an insulating material, typically a clear or transparent plastic, such as polycarbonate, Nylon or the like, or a coated metals. These materials serve as insulators against Radio Frequency (RF) energy from the cutting blade 180, while in the case of clear or transparent plastics, also allowing visibility for the optics of the entire surgical site. The platform 170 is typically a single piece (but could also be formed of multiple pieces fastened together) formed by techniques such as injection molding or the like.

The platform 170 includes a bore 172 for receiving the positioning tube 150, and a groove 174, defined by a lip 176 and sidewall 178, for retaining a cutting blade 180, while it rotates within this groove 174. A gear 182, with teeth 182a, affixed to the shaft 164, at its distal end, engages teeth 184 of the cutting blade 180, typically from inside the cutting blade 180. The gear 182 pushes the cutting blade 180 into the groove 174, and coupled with the groove 174, retains the cutting blade 180 securely in the groove 174. This arrangement allows for rotation of the cutting blade 180, unidirectionally (clockwise or counterclockwise) or bidirectionally (in the directions of the double headed arrow 185), depending on the rotational direction(s) of the motor 212, as detailed below. Alternately, the cutting blade 180 could receive the gear 182 in frictional or magnetic couplings, in order to provide the unidirectional or bidirectional rotation for the cutting blade 180.

The cutting blade 180 is typically in a circular ring shape, although other shapes and configurations, both continuous and non-continuous are also permissible. The cutting blade 180 includes a distal end 186 with an edge 187, typically serrated, the serrations forming the teeth 184. The proximal end 188 of the cutting blade 180 includes a sharpened edge 190 for cutting. This sharpened edge 190 of the cutting blade, that typically rotates at speeds of approximately 1000-5000 rpm, coupled with the retractive movement of the cutting blade 180 (in a direction parallel to the longitudinal axis 123), results in tissue cut into precise tubular pieces, without this tissue having been ripped or torn, and absent turbulence. This provides the optics (in the viewing tube 128) with a clear view of the surgical site. Also, since ripping and tearing is absent here, cut tissue does nor wrap around the cutting unit 160 or positioning tube 150. Moreover, this cutting process generates minimal if any heat, so as not to damage tissues. These precisely cut tissue pieces are suitable for histological studies, upon their removal from the body (detailed below).

Alternately, the cutting edge 190, could be undulating, serrated or other shape, or combinations thereof, so as to provide the above described precise cutting.

The cutting blade 180 is made of a hard material, suitable for conducting RF energy. This hard material is typically a hard, surgical grade metal, such as that used for scalpels and other precision surgical cutting tools, e.g., Stainless Steel, Nitinol, etc.

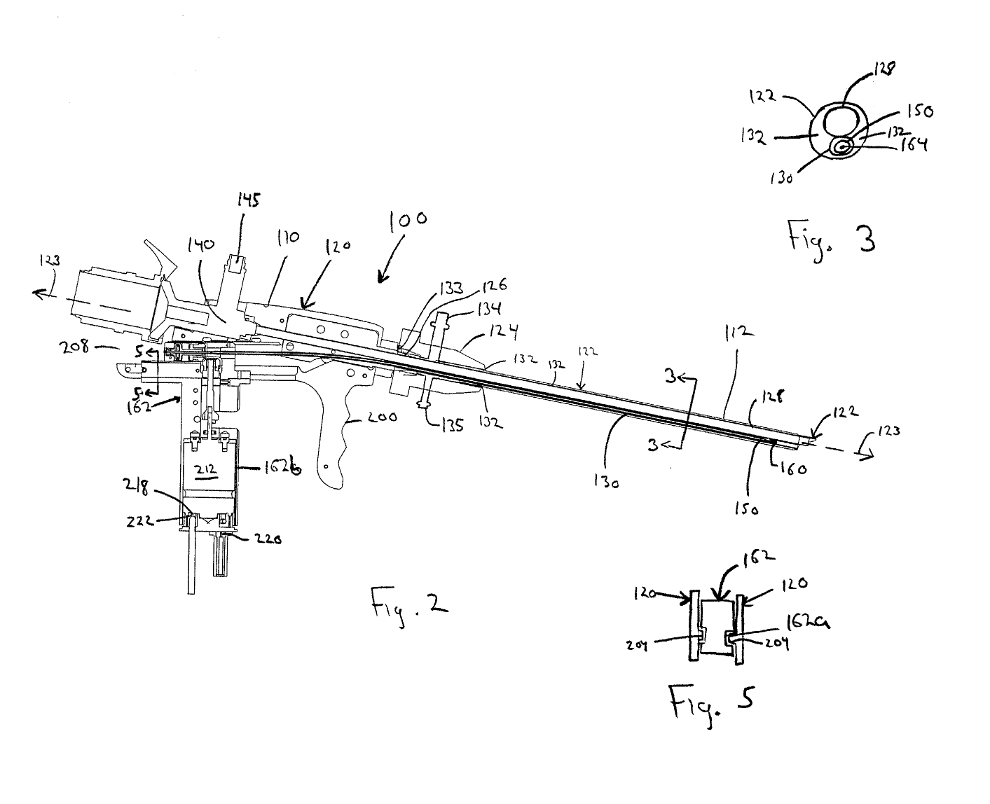

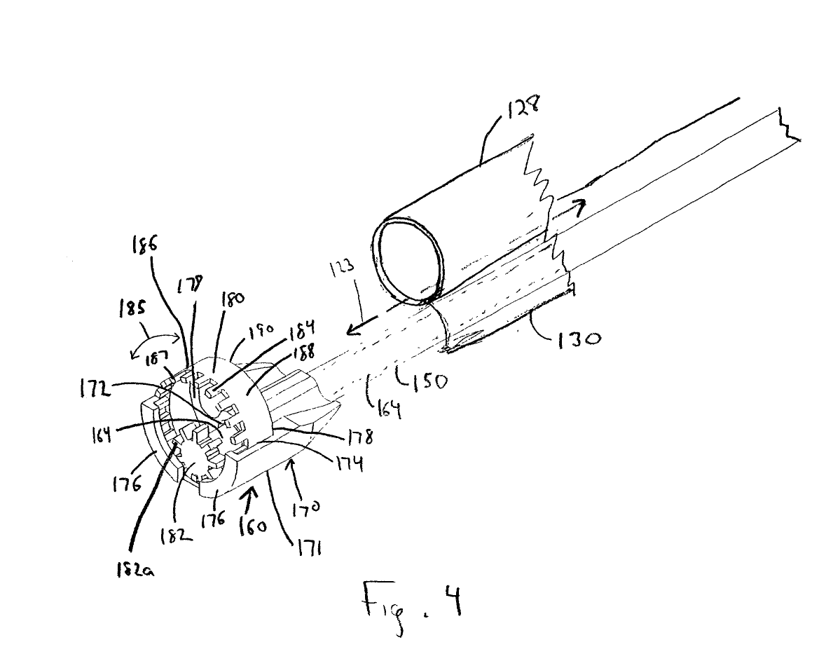

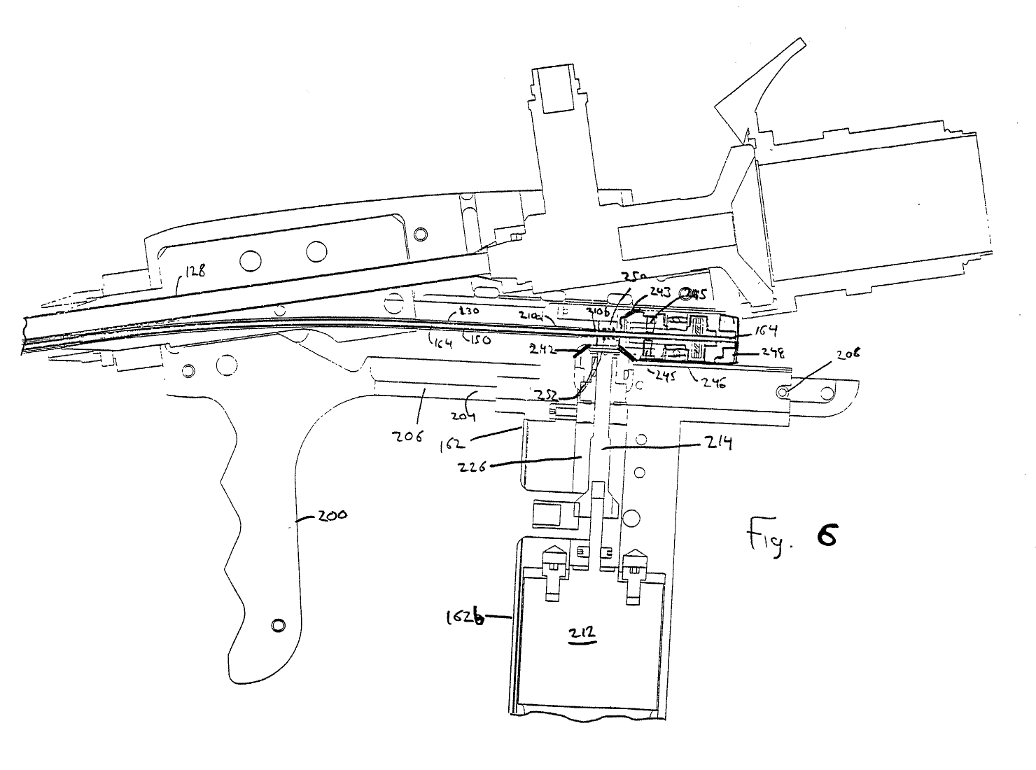

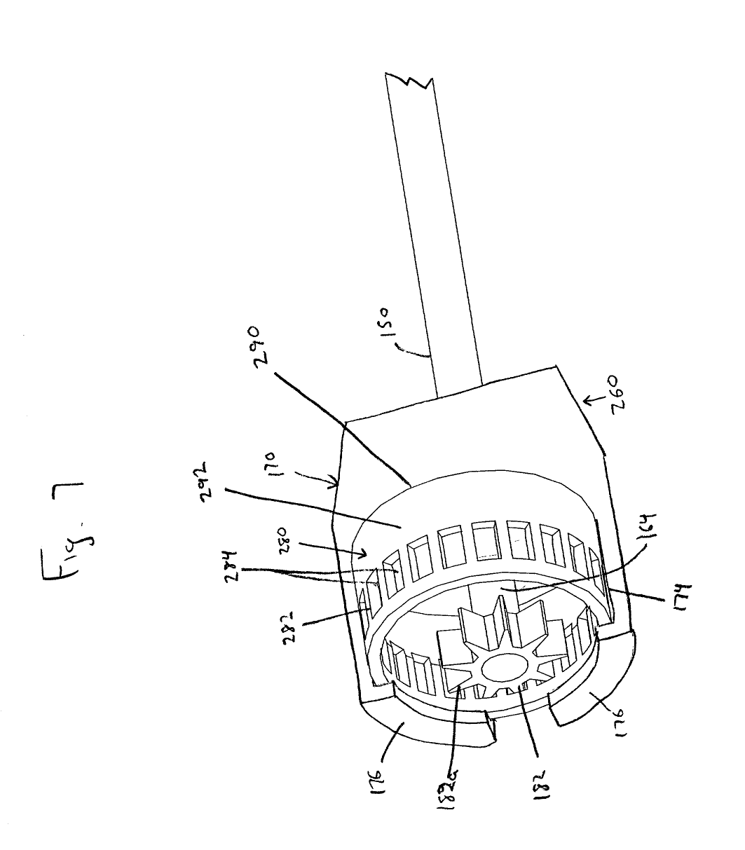

The body 120 includes an integral handle 200 of dimensions suitable for a surgeon's fingers, typically forefinger(s), to grasp it with sufficient control. The frame 162 is slidably mounted on the body 120, as its edges 162a, are “U” shaped, to engage oppositely disposed flanges 204, that form a portion of an opening 206 in the body 120, as shown in FIG. 5. The frame 162 is maintained in this slidable engagement, by a spring 208 (or springs), that allows for movement in directions parallel to the longitudinal axis 123. The spring 208 is biased proximally, so as to keep the frame 162 proximal in the opening 206, whereby the positioning tube 150 and cutting unit 160 remain housed within the sheath 122 when the instrument 100 is in an inactive or non-cutting position.

The frame 162 includes a gripping portion 162b, that is dimensioned to allow a surgeon's thumb to grasp it with sufficient control. By placing his thumb around the gripping portion 162a, the surgeon can slide the frame 162 along the body 120 (in a direction parallel to the longitudinal axis 123, as per the double headed arrow 209), such that the positioning tube 150 and the cutting unit 160 can be moved between distal and proximal positions, when cutting and cauterizing is desired.

Turning now to FIG. 6, the frame 162 is detailed, The frame 162 receives the positioning tube 150, through a first bore portion 210a, that terminates in second bore portion 210b, that is of a larger diameter than that of the shaft 164, but a smaller diameter than that of the positioning tube 150. Accordingly, this second bore portion 210b, serves a stop surface for the positioning tube 150. The positioning tube 150 may be secured in the frame 162 in its fixed position with screws 211 or other adjustable tightening members, as well as adhesives or other conventional fasteners/fastening techniques.

The frame 162 houses a motor 212 that connects to a motor shaft 214, for ultimately rotating the cutting blade 180 of the cutting unit 160. The motor 212 is for example, a 10 Watt motor, capable of rotating unidirectionally (clockwise or counterclockwise) and/or bidirectionally, for example, at speeds of up to 10,000 rpm, with a typical exemplary range for motor speed being approximately 2,500-10,000 rpm.

The motor 212, by its motor shaft 214 connects to the shaft 164 that drives the cutting blade 180 by a gear mechanism illustrated in FIG. 3, and detailed below. The gear mechanism, as well as gear 182, collectively form the gearing for the instrument 100, that, for example, can be set at ratios of approximately 2-2.5, for operation of the instrument 100.

The motor 212 is preferably operated by means of a foot pedal (not shown) that can control the direction and speed of the rotation. The motor also supports an electrical plug 218 that allows an RF generator (not shown) and a DC power supply (not shown) to be attached. The plug 218 has a DC socket 220 and an RF socket 222, as detailed in U.S. patent application Ser. No. 09/593,988.

This connection of the motor 212 to the shaft 164, is for rotating the cutting blade 180. This connection is in accordance with U.S. patent application Ser. No. 09/593,988. Here, motor 212 has its motor shaft 214 that passes through a bearing 226, and terminates in a bevel gear 242. Bevel gear 242 meshes with a corresponding bevel gear 243, that holds the shaft 164 in a fixed engagement, allowing the shaft 164 to rotate, upon rotation of the bevel gears 242, 243. This fixed engagement of the shaft 164 in the bevel gear 243 is maintained with screws 245 or other tightening mechanisms. Shaft 164 passes though the gear 243 and bearing 246. The bearing 246, in turn, is affixed to the frame 162. The shaft 164 terminates in a cap member 248, that is fixed to the frame 162 and receives the shaft 164.

The frame 162 includes a cavity 250 intermediate the bevel gear 243 and the area where positioning tube 150 is affixed to the frame 162. Within this cavity 250 are wires 252, at a tension so as to be in constant contact with the shaft 164, while having enough “play” to move upon rotation of the shaft 164, so as to avoid frictional degradation of the wires 252. The wires 252 terminate in connectors (not shown), that connect to an RF carrier line (not shown) that extends through the frame 162, to the RF socket 222.

This arrangement allows RF power to be provided to the cutting blade 180, typically for cauterization of cut tissue, as the RF energy heats tissue that it contacts. Specifically, the wires 252 contact the shaft 164 that is in conductive contact (by affixation) with the gear 182, that contacts the cutting blade 180, allowing for the transmission of RF energy to the cutting blade 180.

The frame 162 mounts in the body 120, such that when the frame 162 is slid forward, the positioning tube 150, and the shaft 164 also slide forward relative to the sheath 122. The positioning tube 150 and the shaft 164 slide forward together, but only the shaft 164 is free to rotate. The sliding motion of frame 162 continues such that the positioning tube 150 with the cutting unit 160 attached thereto, has placed the cutting unit 160 at the location (surgical site) where the surgeon wishes to remove tissue. Upon activation of the motor and rotation of the cutting blade 180, the positioning tube 150 is retracted, as the surgeon lets the frame 162 move proximally in a controlled manner, by resisting the force provided by the spring 208. This retractive movement, moving the cutting unit 160 proximally, coupled with the rotating cutting blade 180 results in the cutting of precise tissue pieces.

The sheath, tubes, shafts and gears can be constructed out of suitable materials, such as stainless steel, other conventional alloys, hard rubber, plastics, polymeric materials, ceramics or composite materials, such as graphite composites. Flexible polymeric materials can also be used, for the sheaths and tubes. The materials for the sheath, tubes and shaft can be fully or partly coated with polymeric materials and other plastics, such as Teflon® or other known materials, to provide for lubrication to aid in insertion and to provide electrical, mechanical and fluid isolation. In particular, shaft 164, and gear 182, should be made of an RF conducting material.

The body 120 is typically made from a hard polymeric material, typically injection molded in pieces or shells, that are joined by mechanical fasteners, such as screws, adhesives, welds or the like. The remainder of the instrument 100 can also be constructed of stainless steel or polymeric materials.

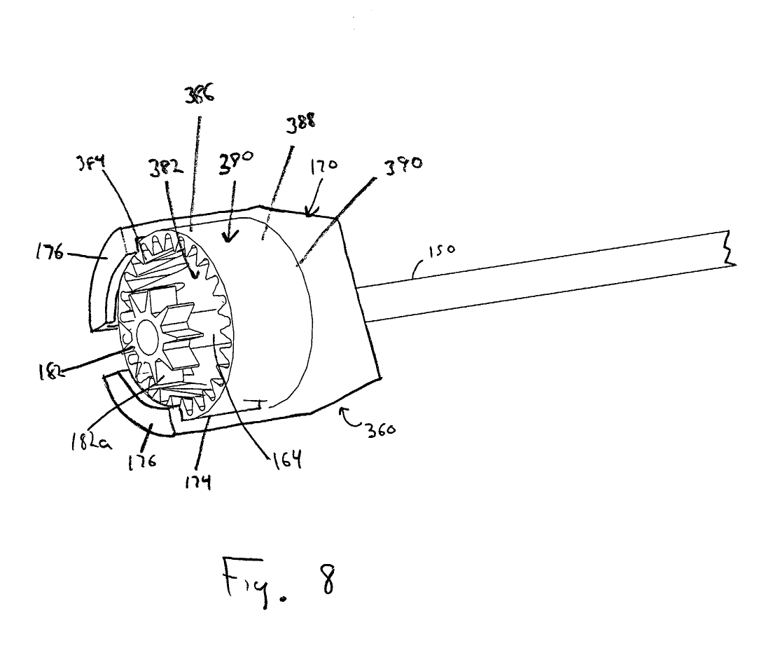

FIG.7 details a second embodiment of the cutting unit 260. This cutting unit 260 is of similar components and arrangement to those of cutting unit 160, as shown (for example in FIG. 4) and detailed above (similar elements are incremented by “100”), except where indicated. Here, there is a cutting blade 280, that includes a distal end 282 that includes openings 284. The cutting edge 290 is on the proximal end 292 of the cutting blade 280. The gear 182 is positioned such that it is inside of the cutting blade 280, and its teeth 182a engage the correspondingly configured openings 284 of the cutting blade 280. Upon rotation of the gear 182, this engagement of the gear teeth 182a in the openings 284 causes rotation of the cutting blade 280.

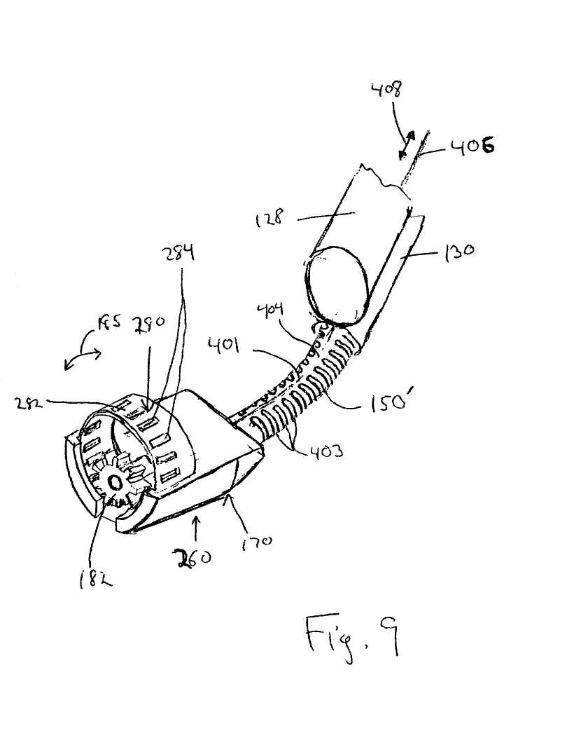

FIG. 8 details a third embodiment of the cutting unit 360. This cutting unit 360 is of similar components and arrangement to those of cutting unit 160, as shown (for example in FIG. 4) and detailed above (similar elements are incremented by “200”), except where indicated. Here, there is a cutting blade 380, that includes an inner side 382 with inwardly protruding teeth 384, extending upward to the distal end 386 of the cutting blade 380. The proximal end 388 of 30 the cutting blade 380 includes a sharpened cutting edge 390. The gear 182 is positioned with respect to the shaft 164, so as to have at least portion of the gear 182, and typically all of it, inside the cutting blade 380, where its teeth 182a engage the respective spaces 392 between the inwardly protruding teeth 384. Upon rotation of the gear 182, this engagement of teeth 182a, 384 allow for rotation of the cutting blade 380.

An exemplary operation of the instrument 100 with cutting unit 160 will now be described. This description is exemplary only as any of the cutting units 260, 360 could also be used as detailed herein. In operation, the surgical site, e.g., the prostate, is accessed by the instrument 100, as the sheath 122 enters the urethra. When cutting is desired, the surgeon moves the frame 162 forward, such that the positioning tube 150 with the cutting unit 160 extends distally, out of the sheath 122, to a desired point beyond (distal) to the sheath 122. The instrument 100 is now in an active or cutting position, as it is typically in contact with tissue. The motor is now activated, rotating the cutting blade 180 unidirectionally. The cutting unit 160, with the cutting blade 180 is now moved proximally, toward the body 120 of the instrument 100, as the surgeon allows the frame 162 to move proximally, as per the spring biasing, in a controlled manner. This proximal movement of the cutting blade 180, coupled with its rotation, cuts tissue in precise pieces, typically tubular in shape, while generating minimal, if any heat. By not generating heat upon cutting, the risk of damage to tissues surrounding the prostate and potential impotence is minimized. Cutting in this manner can continue for as long as desired, as the surgeon manipulates the instrument 100 to the desired cutting locations.

Throughout the process, irrigation fluid is transported through the lumen 132. The irrigation fluid outflow from the sheath 122, serves as a carrier for the cut tissue, flushing it into the bladder, so as to be removed from the surgical site. Since the cut tissue is flushed away from the cutting unit 160 and viewing tube 128, the surgeon views the entire process clearly through the optics in the viewing tube 128.

The cut tissue can also be evacuated from the surgical site by suction (aspiration). Suction can be through the lumen 132 of the instrument 100, provided a suction tube is attached to one of the ports 134, 135. Alternately, a suction tube can be inserted through one of the ports 134, 135 and through the lumen and moved distally, beyond the cutting blade 180, to allow forward flow of the fluid and cut tissue, while capturing the cut tissue for aspiration at a point proximate the cutting blade. This allows for maintaining a clear view of the surgical site.

In another alternate embodiment, a suction tube can be placed proximate, typically distal, to the cutting blade, by accessing the surgical site through the bladder. This can be done by typical accessing techniques, such as with trocars, or other puncturing or needle type instruments.

Once cutting is concluded, or after a cut has been made, the RF energy source is activated, whereby the cutting blade 180, with RF energy can be placed into contact with the desired, typically bleeding tissue. The cutting blade 180, as a result of receiving the RF energy, has now heated instantaneously, such that bleeding tissue can be contacted with the cutting blade 180, cauterizing it. The remaining portion of the cutting unit 160 can now be retracted into the sheath 122, such that the cutting unit is now in an inactive or non-cutting position, and can be removed from the urethra.

The cut tissue pieces, if not removed by suction, and now in the bladder, can be removed by standard bladder flushing procedures.

In another alternate embodiment, as shown in FIG. 9, the instrument 100 (shown and described above) can have a steerable positioning tube 150′. The cutting unit 260 (shown in FIG. 7 and described above) is exemplary of cutting units. However, any other of the disclosed cutting units 160, 360 are also suitable for use with this embodiment.

This steerable positioning tube 150′ includes a segment 401, typically at the distal end of the positioning tube 150′ proximate the cutting unit 160, that includes cuts 403 therein. The cuts 403 are between stiffeners 404 (only one shown). These cuts 403 and stiffeners 404 allow for this segment 401 to be steered in directions lateral to the cuts 403. The segment 401 is typically moved by a wire 406 (that moves the segment 401 by being pulled in the direction of the double headed arrow 408) or other motion translating structure, that is received in a steering mechanism in the body 120. This steering mechanism may be for example, in accordance with the steering mechanism detailed in the Storz® Flexible Pediatric Cystoscope, Model No. 11274, and the Storz® Flexible Vretro-Fiberscope, Model No. M274 AA.

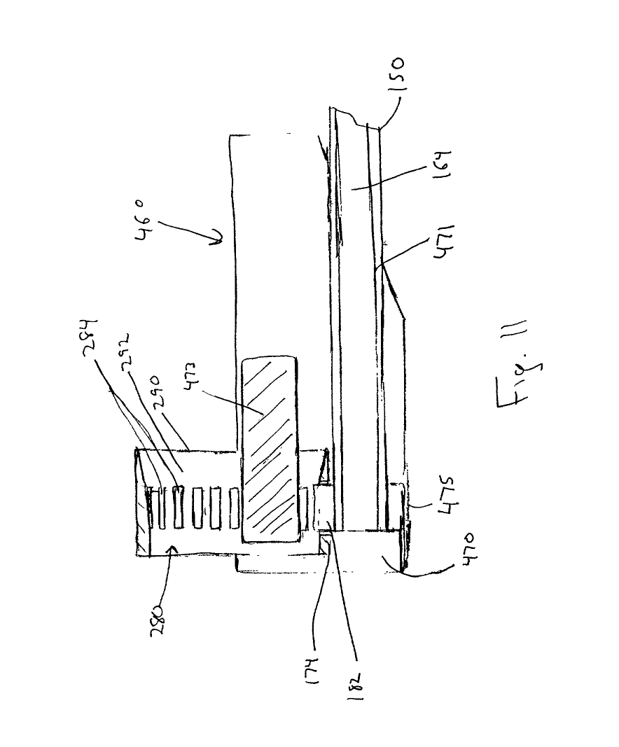

Another alternate embodiment of the invention is shown in FIGS. 10 and 11. The instrument 100 (shown and described above) has a cutting unit 460, that is similar to cutting unit 260 (FIG. 7, shown and described above), with similar components, except where indicated. Specifically, cutting unit 460 differs from cutting unit 260 in that the cutting blade 280 is driven externally by the gear 182 (gear teeth 182a engage openings 284 in the cutting blade 280 from outside of the cutting blade 280), rather than internally. Accordingly, the platform 170 includes a cut out portion 470 for receiving the gear 182 and a bore 471 for receiving the shaft, as well as pins 473 (single or multiple), inside of the cutting blade 280, for retaining the cutting blade 280 in the groove 174. Curvature of the outer side 475 of the platform 470 is such that the platform 470 conforms within the curvature of the sheath 122.

In other alternate embodiments, cutting units 160 and 360 could be easily modified, as detailed here, to operate in accordance with cutting unit 460.

In another alternate embodiment, the cutting blades 180, 280, 380, could also be non-continuous (although the continuous cutting blades 180, 280, 380 detailed above can also be used in this embodiment). Here, the motor 212 is configured such that it rotates the cutting blades (via the shaft 164) bidirectionally, in arc portions, less than that of a full 360 degree arc, typically approximately 90 degrees from the vertical, so that the cutting blades rotate in a back and forth (pendulum-like) manner, at speeds suitable for the above-detailed precise cutting.

Alternate embodiments of the instrument 100 may be designed such that cutting is in the distal direction, away from the instrument 100. In such an instrument, construction and arrangement of elements is similar to those detailed for instrument 100 above, except that the orientation of the cutting blade 180 is switched (cutting edge 190 is now the distal end) and accordingly, the shaft 164 is shortened such that the gear 182 can engage the respective portions, now at the proximal end of the cutting blade. Similarly, cutting units 260, 360 and 460 could be modified as detailed here, to operate in this manner.

Although several exemplary preferred embodiments of the invention have been disclosed, it will be apparent to those skilled in the art that various changes and modifications can be made which will achieve some of the advantages of the invention without departing from the spirit and scope of the invention. For example, it will be obvious to those reasonably skilled in the art that elements and configurations thereof are exemplary, and other equivalent elements and configurations thereof can be used with the same effect. Other aspects, such as the specific mechanical configuration of the instrument, as well as other modifications to the inventive concept are intended to be covered by the appended claims.

What is claimed is:

1. A surgical instrument for cutting tissue comprising:
a positioning tube having a longitudinal axis;
a cutting blade at least substantially continuous;
a shaft including a proximal end and a distal end, said distal end of said shaft rotatably engaging said cutting blade, at least a portion of said shaft housed within said positioning tube, and
a frame configured for receiving said proximal end of said shaft such that said shaft can rotate therein, and configured for holding said proximal end of said shaft therein, such that movement of said frame along said longitudinal axis moves said shaft and said cutting blade along said longitudinal axis.

2. The surgical instrument of claim 1, wherein said positioning tube is attached to said frame, such that movement of said frame along said longitudinal axis moves said shaft and said positioning tube together along said longitudinal axis.

3. The surgical instrument of claim 2, additionally comprising a platform, said platform attached to said positioning tube and configured for supporting at least a portion of said cutting blade.

4. The surgical instrument of claim 3, wherein said cutting blade includes a continuous ring.

5. The surgical instrument of claim 4, additionally comprising a gear, said gear attached to said shaft.

6. The surgical instrument of claim 5, wherein said gear extends at least partially into said cutting blade, and said gear and said cutting blade including correspondingly configured portions for temporarily engaging each other, so as to permit rotation of said cutting blade upon rotation of said shaft.

7. The surgical instrument of claim 6, wherein said gear includes protruding teeth.

8. The surgical instrument of claim 7, wherein said cutting blade includes a first end and a second end, said first end including a cutting edge.

9. The surgical instrument of claim 8, wherein said second end includes an edge, and said correspondingly configured portions include serrations along said edge.

10. The surgical instrument of claim 6, wherein said correspondingly configured portions include openings in said cutting blade.

11. The surgical instrument of claim 10, wherein said cutting blade includes a first end and a second end, said first end including a cutting edge and said second end including said openings.

12. The surgical instrument of claim 6, wherein said correspondingly configured portions include inwardly protruding teeth.

13. The surgical instrument of claim 12, wherein said cutting blade includes a first end and a second end and an inner side, said first end including a cutting edge and said second end including said inwardly protruding teeth.

14. The surgical instrument of claim 1, additionally comprising a Radio Frequency (RF) energy transport system comprising:
at least one wire in said frame for transporting RF energy, said wire in conductive communication with said shaft.

15. The surgical instrument of claim 3, additionally comprising a body configured for receiving at least a portion of said frame in a slidable engagement.

16. The surgical instrument of claim 15, additionally comprising a tubular sheath for attachment to said body, and said shaft, positioning tube and cutting blade are configured for movement along said longitudinal axis between positions where said cutting blade is inside of said tubular sheath and outside of said tubular sheath.

17. The surgical instrument of claim 1, wherein said shaft is flexible.

18. The surgical instrument of claim 17, wherein said positioning tube is flexible.

19. The surgical instrument of claim 18, wherein said positioning tube is configured for control by a steering mechanism.

20. A method for cutting tissue comprising:
providing a surgical instrument comprising:
a tubular sheath having a longitudinal axis; and
a rotatable ring defining a cutting blade, said ring mounted with respect to said tubular sheath so as to be movable in directions along said longitudinal axis between positions inside said tubular sheath and outside of said tubular sheath;
accessing a surgical site;
moving said cutting blade out of said tubular sheath;
rotating said cutting blade; and
moving said cutting blade toward said tubular sheath, while said cutting blade is rotating.

21. The method of claim 20, additionally comprising irrigating said surgical site by providing fluid flowing in a direction away from said tubular sheath.

22. The method of claim 20, wherein accessing said surgical site includes entering the male urethra with said tubular sheath and moving tubular sheath to a position at least proximate the prostate.

23. The method of claim 20, additionally comprising, contacting tissue with said cutting blade and activating radio frequency (RF) energy, such that said RF energy moves to said cutting blade for cauterizing said tissue upon contact with said cutting blade.

24. The method of claim 20, additionally comprising steering said cutting blade.
